(19) United States
(12) Patent Application Publication
(10) Pub. No.: US 2025/0209938 A1
(43) Pub. Date: Jun. 26, 2025

(54) Apparatus for Evaluating Virtual Surgery

(71) Applicants: Kyu Eun Lee, Seoul (KR); Hyoun-Joong Kong, Seoul (KR); Eun Jung Lee, Seoul (KR); Hoorang Shin, Jeju-si (KR)

(72) Inventors: Kyu Eun Lee, Seoul (KR); Hyoun-Joong Kong, Seoul (KR); Eun Jung Lee, Seoul (KR); Hoorang Shin, Jeju-si (KR)

(21) Appl. No.: 18/397,164

(22) Filed: Dec. 27, 2023

(30) Foreign Application Priority Data
Dec. 20, 2023 (KR) 10-2023-0187711

(51) Int. Cl. G09B 23/28; G09B 23/30

(52) U.S. Cl. CPC G09B 23/285; G09B 23/30

(57) ABSTRACT

Disclosed is an apparatus for evaluating a virtual surgery based on an evaluation index. The present embodiment provides an apparatus for evaluating a virtual surgery based on an evaluation index, which is capable of: generating an evaluation index based on an average point of three-dimensional coordinates which are obtained through a repeated performance of a virtual simulation by an operating surgeon (evaluator) with a high surgical skill level; and extracting the three-dimensional coordinates obtained in a process of performing the virtual simulation by a surgeon-in-training (subject) with a low surgical skill level and comparing the same with the above-generated evaluation index so as to evaluate the virtual surgery performed by the surgeon-in-training.

### BACKGROUND OF THE INVENTION

### 1. Field of the Invention

One embodiment of the present invention relates to an apparatus for evaluating a virtual surgery based on an evaluation index.

### 2. Description of the Prior Art

The content described below simply provides background information related to the present embodiment and does not constitute the related art.

Surgeons generally undertake an extensive study before performing a surgical procedure. In general, surgeons conduct a study using anatomical models which include photographs and pictures. In recent years, various pre-operative diagnostic procedures (e.g., x-rays, CT, MRI, etc.) have used patient-specific anatomical information.

It is preferable that surgeons use additional anatomical and surgical procedure information. A surgeon who plans a surgery for a particular patient is provided with a video record on a surgical site for a previous surgical procedure performed on that particular patient. One or more surgical video records on surgical procedures for other patients similar to the planned surgical procedure for that particular patient are provided to the surgeon. Such information is provided to the surgeon before the surgeon starts a specific surgical procedure. The above-described information is intraoperatively provided to the surgeon.

It is educationally important to repeatedly practice and thoroughly know the process of the corresponding surgery, which is divided step by step, and thus a virtual surgery environment is provided and a simulated virtual content is provided so that the process of the same surgery may be divided for each step and followed.

### SUMMARY OF THE INVENTION

An object of the present embodiment is to provide an apparatus for evaluating a virtual surgery based on an evaluation index, which is capable of: generating an evaluation index based on an average point of three-dimensional coordinates which are obtained through a repeated performance of a virtual simulation by an operating surgeon (evaluator) with a high surgical skill level; and extracting the three-dimensional coordinates obtained in a process of performing the virtual simulation by a surgeon-in-training (subject) with a low surgical skill level and comparing the same with the above-generated evaluation index so as to evaluate the virtual surgery performed by the surgeon-in-training.

According to one aspect of the present embodiment, there may be provided an apparatus for evaluating a virtual surgery, which includes: an evaluator simulation data acquisition unit for obtaining an evaluator's evaluator virtual surgery simulation data from a first MR device; an evaluation index table generation unit for extracting a preset evaluator-used surgical instrument parameter for each surgical instrument used by the evaluator based on the evaluator virtual surgery simulation data, and generating an evaluation index table based on the used surgical instrument parameter; a subject simulation data acquisition unit for obtaining a subject's subject virtual surgery simulation data from a second MR device; and a virtual surgery evaluation unit for extracting a preset subject-used surgical instrument parameter for each surgical instrument used by the subject based on the subject virtual surgery simulation data, and comparing the evaluation index table with the subject-used surgical instrument parameter to evaluate a subject's skill level.

As described above, according to the present embodiment, there is an effect of: generating an evaluation index based on an average point of three-dimensional coordinates which are obtained through a repeated performance of a virtual simulation by an operating surgeon (evaluator) with a high surgical skill level; and extracting the three-dimensional coordinates obtained in a process of performing the virtual simulation by a surgeon-in-training (subject) with a low surgical skill level and comparing the same with the above-generated evaluation index so as to evaluate the virtual surgery performed by the surgeon-in-training.

### BRIEF DESCRIPTION OF THE DRAWINGS

FIG. 1 is a view showing a system for evaluating a virtual surgery based on an evaluation index according to the present embodiment.

FIG. 2 is a view schematically showing an apparatus for evaluating a virtual surgery according to the present embodiment.

FIG. 3 is a flowchart for explaining a method for evaluating a virtual surgery according to the present embodiment.

FIGS. 4A, 4B, and 4C are exemplary views showing a preparation for virtual surgery using an MR device according to the present embodiment.

FIGS. 5A and 5B are views showing an evaluation index table according to the present embodiment.

FIG. 6 is an exemplary view of comparing an evaluation index table according to the present embodiment with a subject-used surgical instrument parameter.

FIGS. 7A, 7B, 7C, 7D, 7E, 7F, 7G, 7H, and 7I are exemplary views showing a virtual surgery using a device according to the present embodiment.

### DETAILED DESCRIPTION OF THE INVENTION

Hereinafter, the present embodiment will be described in more detail with reference to the accompanying drawings.

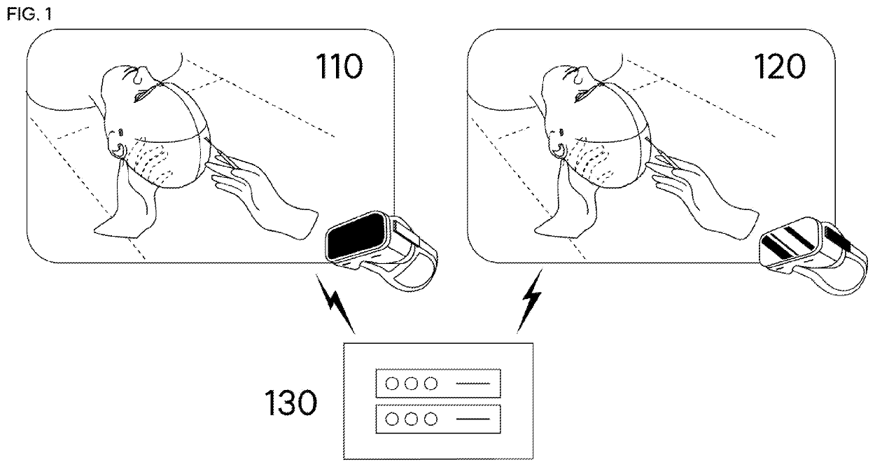

FIG. 1 is a view showing a system for evaluating a virtual surgery based on an evaluation index according to the present embodiment.

The system for evaluating a virtual surgery based on an evaluation index according to an embodiment of the present invention may include a first MR device 110, a second MR device 120, and a virtual surgery evaluation apparatus 130. Components included in the virtual surgery evaluation system may not be necessarily limited thereto.

The first and second mixed reality (MR) devices 110 and 120 may refer to an apparatus for realizing and providing mixed reality combining the real world and virtual reality. The first and second MR devices 110 and 120 may be mainly implemented in the form of headsets or glasses.

The first and second MR devices 110 and 120 may provide mixed reality using at least one or more functions of transparent display, space recognition technology, gesture or voice control, virtual object and information display, sensor and tracking system. The first and second MR devices 110 and 120 may be used for various purposes in a medical field, and may be particularly often applied in a surgical field.

The first and second MR devices 110 and 120 may allow medical professionals to receive virtual reality surgery training for education and training using virtual reality. The first and second MR devices 110 and 120 may allow the medical professionals to receive training in various scenarios before actual surgery, so as to develop better surgical techniques and learn new techniques or procedures.

The first and second MR devices 110 and 120 may allow doctors to visualize a patient's anatomical structure in real time in 3D using 3D visualization and navigation functions. The first and second MR devices 110 and 120 may identify an accurate position during a surgery, obtain information on an important structure, increase accuracy, and secure safety during the surgery through the patient's anatomical 3D structure.

The first and second MR devices 110 and 120 may provide real-time intervention and augmented information so that doctors may check the patient's data, images, biological signals, and the like in real time during surgery. The first and second MR devices 110 and 120 may immediately provide important information to the doctors during surgery to assist in decision making and improve the accuracy of the surgery.

The first and second MR devices 110 and 120 may enable remote medical collaboration so that medical professionals may share the patient's condition and collaborate with each other in real time even when medical professionals are geographically separated from each other.

The first MR device 110 may enter an evaluation index table generation mode in a virtual environment according to an operation or command of an evaluator (an operating surgeon with a high surgical skill) who is equipped with the device. The first MR device 110 may receive an input of a surgery name and a surgical instrument to be measured for skill level according to an operation or command of an evaluator (an operating surgeon with a high surgical skill) who is equipped with the device, and then may grip a displayed virtual object using a virtual hand gesture.

The first MR device 110 may perform a simulation action to be evaluated according to an operation or command of an evaluator (an operating surgeon with a high surgical skill) who is equipped with the device through a hand gesture, and receive the completion of a first time.

The first MR device 110 may attempt to repeat the same simulation action n times according to an operation or command of an evaluator (an operating surgeon with a high surgical skill) who is equipped with the device, and input the completion of measurement for generating an evaluation index table.

The first MR device 110 may enter an evaluation index table generation mode in a virtual environment by an evaluator's operation or command, output a target surgery name for generating the evaluation index table and a virtual object corresponding to a surgical instrument, and allow the surgical instrument to be gripped using a virtual hand gesture.

The first MR device 110 may generate the evaluator virtual surgery simulation data when a corresponding surgery is completed after allowing each surgical procedure to be performed by hand gesture according to a simulation action of the surgical instrument while an anatomical structure of a patient corresponding to the surgery name is visualized.

The second MR device 120 may perform a surgery in a virtual environment according to an operation or command of a subject (a surgeon-in-training with a low surgical skill) who is equipped with the device.

The second MR device 120 may enter a skill level evaluation mode in a virtual environment by the subject's operation or command, output a target surgery name for evaluating a skill level and a virtual object corresponding to the surgical instrument, and allow the surgical instrument to be gripped using a virtual hand gesture.

The second MR device 120 may generate the subject virtual surgery simulation data when a corresponding surgery is completed after allowing each surgical procedure to be performed by hand gesture according to a simulation action of the surgical instrument while an anatomical structure of a patient corresponding to the surgery name is visualized.

A virtual surgery evaluation apparatus 130 according to the present embodiment may obtain evaluator virtual surgery simulation data of an operating surgeon (evaluator) with a high surgical level from the first MR device 110.

The virtual surgery evaluation apparatus 130 may extract, from the evaluator virtual surgery simulation data, a preset evaluator-used surgical instrument parameter (e.g., at least one or more of a surgical instrument's entry position (center point), entry angle, entry direction, speed, delay time, and action) for each surgical instrument used by the operating surgeon in an image.

The virtual surgery evaluation apparatus 130 may generate a virtual simulation evaluation index for education based on a plurality of evaluator-used surgical instrument parameters (e.g., at least one or more of a surgical instrument's entry position (center point), entry angle, entry direction, speed, delay time, and action).

The virtual surgery evaluation apparatus 130 may calculate a center point by x, y, and z values of each coordinate of the surgical instrument for each surgical procedure based on the input evaluator virtual surgery simulation data, and calculate an average center point, an average entry angle/direction, an average speed, and an average delay time by dividing the resulting center point by a total number of coordinates (n times).

The virtual surgery evaluation 130 apparatus may automatically set a threshold based on a value farthest from a center value to automatically divide a differential evaluation index table.

The virtual surgery evaluation apparatus 130 may generate a center point virtual action of a perfect value based on calculation results, and may automatically generate an evaluation index table with n points based on the center point virtual action of the perfect value. The virtual surgery evaluation apparatus 130 may enable the evaluator to modify and reflect a weight of evaluation items in the automatically generated evaluation index table, and may finally complete the evaluation index table.

The virtual surgery evaluation apparatus 130 according to the present embodiment may evaluate a virtual surgery simulation of a surgeon-in-training (a subject) with a low surgical skill level based on the evaluation index table. The second MR device 120 may perform a surgery in a virtual environment according to an operation or command of a subject (a surgeon-in-training with a low surgical skill) who is equipped with the device.

The virtual surgery evaluation apparatus 130 may obtain subject virtual surgery simulation data about a surgeon-in-training (a subject) with a low surgical skill from the second MR device 120. The virtual surgery evaluation apparatus 130 may perform a virtual simulation evaluation on the subject based on the evaluation index table.

The virtual surgery evaluation apparatus 130 may classify the subject virtual surgery simulation data according to a surgical process, extract n points previously generated for each process, and automatically evaluate a score of the subject in accordance with the evaluation index table. The virtual surgery evaluation apparatus 130 may allow the subject to compare his or her action with a perfect point action (guide overlay).

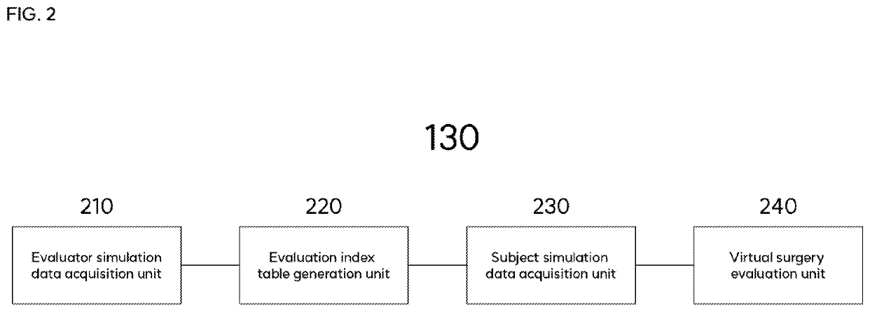

FIG. 2 is a view schematically showing an apparatus for evaluating a virtual surgery according to the present embodiment.

The virtual surgery evaluation apparatus 130 according to the present embodiment may include an evaluator simulation data acquisition unit 210, an evaluation index table generation unit 220, a subject simulation data acquisition unit 230, and a virtual surgery evaluation unit 240. Components included in the virtual surgery evaluation apparatus 130 may not be necessarily limited thereto.

Each component included in the virtual surgery evaluation apparatus 130 may be connected to a communication path connecting a software module or a hardware module inside the apparatus and may be organically operated with each other. These components may communicate using one or more communication buses or signal lines.

Each component of the virtual surgery evaluation apparatus 130 shown in FIG. 2 may refer to a unit in which at least one function or operation is processed, and may be implemented as a software module, a hardware module, or a combination of software and hardware.

The evaluator simulation data acquisition unit 220 may obtain evaluator virtual surgery simulation data for the evaluator from the first MR device 110.

The evaluation index table generation unit 220 may extract a preset evaluator-used surgical instrument parameter for each surgical instrument used by the evaluator based on the evaluator virtual surgery simulation data, and generate an evaluation index table based on the subject-used surgical instrument parameter.

The evaluation index table generation unit 220 may generate the evaluation index table based on the evaluator-used surgical instrument parameter by recognizing that data for generating an evaluation table for a virtual surgery is accumulated when a corresponding surgery is repeated by a preset number of times (n times) after allowing each surgical procedure to be performed on a simulation action for the target surgery name which is identical to the operation or command of the evaluator from the first MR device 110.

The evaluation index table generation unit 220 may divide the evaluator virtual surgery simulation data into a plurality of the evaluator-used surgical instrument steps, and extract parameter including at least one or more of a surgical instrument's entry position (center point), entry angle, entry direction, speed, delay time, and action for each surgical instrument used by the evaluator in each step.

The evaluation index table generation unit 220 may generate the evaluation index table based on an average value for at least one or more of a surgical instrument's entry position (center point), entry angle, entry direction, speed, delay time, and action for each surgical instrument used by the evaluator in each step.

The evaluation index table generation unit 220 may generate an evaluation index table based on an average value of preset surgical instrument-related parameters.

The evaluation index table generation unit 220 may calculate a center point by x, y, and z values of each coordinate of the surgical instrument for each surgical procedure based on the input evaluator virtual surgery simulation data, and calculate an average center point, an average entry angle/direction, an average speed, and an average delay time by dividing the resulting center point by a total number of coordinates (n times).

The evaluation index table generation unit 220 may generate a center point virtual action of a perfect value based on calculation results, and may automatically generate an evaluation index table with n points based on the center point virtual action of the perfect value.

The evaluation index table generation unit 220 may enable the evaluator to modify and reflect a weight of evaluation items in the automatically generated evaluation index table, and may finally complete the evaluation index table.

The subject simulation data acquisition unit 230 may obtain subject virtual surgery simulation data for the subject from the first MR device 120.

The virtual surgery evaluation unit 240 may extract a preset subject-used surgical instrument parameter for each surgical instrument used by the subject based on the subject virtual surgery simulation data, and compare the evaluation index table with the subject-used surgical instrument parameter to evaluate a subject's skill level. The virtual surgery evaluation unit 240 may compare with the subject-used surgical instrument parameter based on the previously calculated evaluation index table so as to determine the subject's skill level.

The virtual surgery evaluation unit 240 may classify the subject virtual surgery simulation data according to a surgical process, extract n points previously generated for each process, and automatically evaluate a score of the subject according to the evaluation index table. The virtual surgery evaluation unit 240 may allow the subject to compare his or her action with a perfect point action (guide overlay).

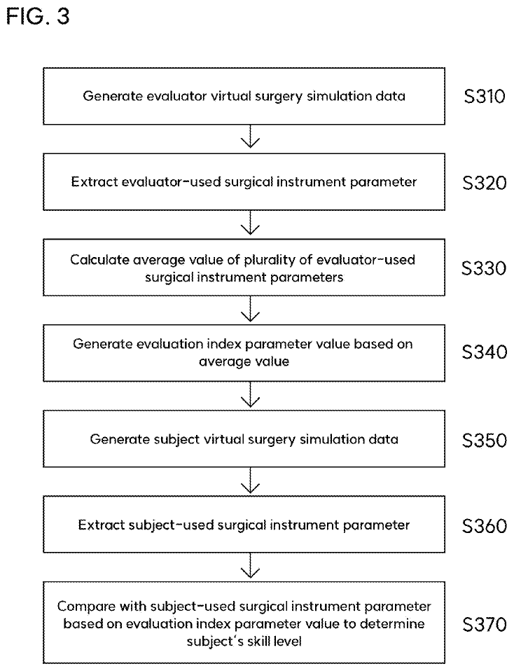

FIG. 3 is a flowchart for explaining a method for evaluating a virtual surgery according to the present embodiment.

The first MR device 110 may generate the evaluator virtual surgery simulation data for the operating surgeon (evaluator) with a high skill level while a patient's anatomical structure is visualized (S310).

The virtual surgery evaluation apparatus 130 may extract, from the evaluator virtual surgery simulation data obtained from the first MR device 110, a preset evaluator-used surgical instrument parameter (e.g., at least one or more of a surgical instrument's entry position (center point), entry angle, entry direction, speed, delay time, and action) for each surgical instrument used by the operating surgeon in an image (S320).

The virtual surgery evaluation apparatus 130 may calculate each average value of a plurality of evaluator-used surgical instrument parameters (e.g., at least one or more of a surgical instrument's entry position (center point), entry angle, entry direction, speed, delay time, and action). In S330, the virtual surgery evaluation apparatus 130 may calculate an overall score by assigning different weights to each parameter. When the virtual surgery evaluation apparatus 130 may not only calculate an average value, but also determine a threshold range by a value closest to the average and a value farthest from the average, when an experienced operating surgeon uses a plurality of specific surgical instruments.

The virtual surgery evaluation apparatus 130 may generate an evaluation index table based on an average value of preset surgical instrument-related parameters (S340). In S340, the virtual surgery evaluation apparatus 130 may calculate a center point by x, y, and z values of each coordinate of the surgical instrument for each surgical procedure based on the input evaluator virtual surgery simulation data, and calculate an average center point, an average entry angle/direction, an average speed, and an average delay time by dividing the resulting center point by a total number of coordinates (n times). The virtual surgery evaluation apparatus 130 may generate a center point virtual action of a perfect value based on calculation results, and may automatically generate an evaluation index table with n points based on the center point virtual action of the perfect value. The virtual surgery evaluation apparatus 130 may enable the evaluator to modify and reflect a weight of evaluation items in the automatically generated evaluation index table, and may finally complete the evaluation index table.

The second MR device 120 may generate the subject virtual surgery simulation data for a surgeon-in-training (subject) while a patient's anatomical structure is visualized (S350).

The virtual surgery evaluation apparatus 130 may extract, from the subject virtual surgery simulation data obtained from the second MR device 120, a preset subject-used surgical instrument parameter (e.g., at least one or more of a surgical instrument's entry position (center point), entry angle, entry direction, speed, delay time, and action) for each surgical instrument used by the subject in an image (S360).

The virtual surgery evaluation apparatus 130 may compare with the subject-used surgical instrument parameter based on the previously calculated evaluation index table so as to determine the subject's skill level (S370). In S370, the virtual surgery evaluation apparatus 130 may classify the subject virtual surgery simulation data according to a surgical process, extract n points previously generated for each process, and automatically evaluate a score of the subject according to the evaluation index table. The virtual surgery evaluation apparatus 130 may allow the subject to compare his or her action with a perfect point action (guide overlay).

FIG. 3 shows that S310 to S370 are sequentially performed, but not limited thereto. In other words, the steps shown in FIG. 3 may be applied in such a way that the steps are modified or one or more steps are performed in parallel, and thus FIG. 3 is not limited to a time series order.

As described above, a method for evaluating a virtual surgery according to the present embodiment shown in FIG. 3 may be implemented as a program and recorded in a computer-readable recording medium. A recording medium in which a program for implementing a virtual surgery evaluation method according to the present embodiment and is readable by a computer may include all types of recording devices in which data readable by a computer system are stored.

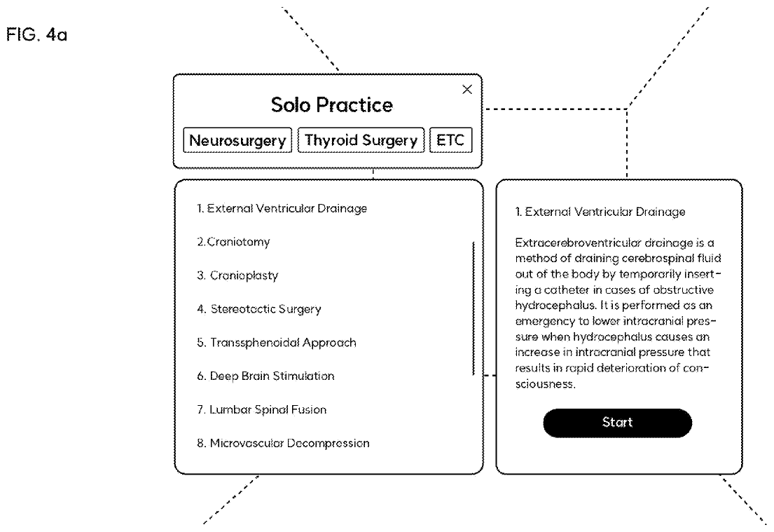

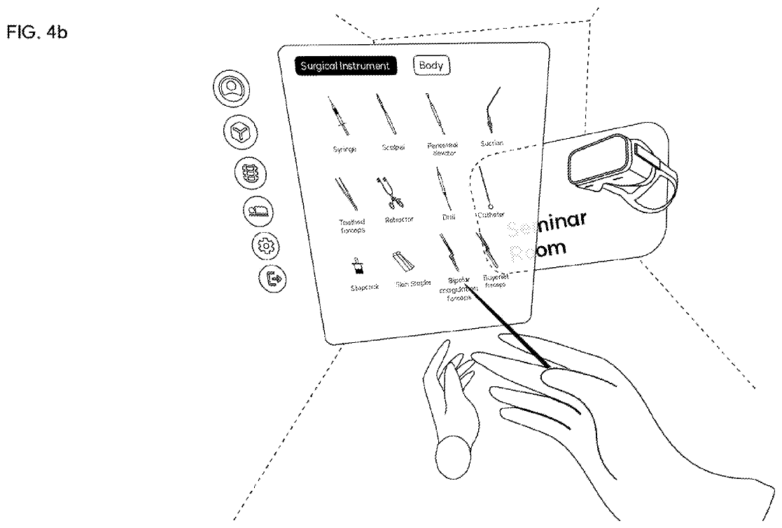

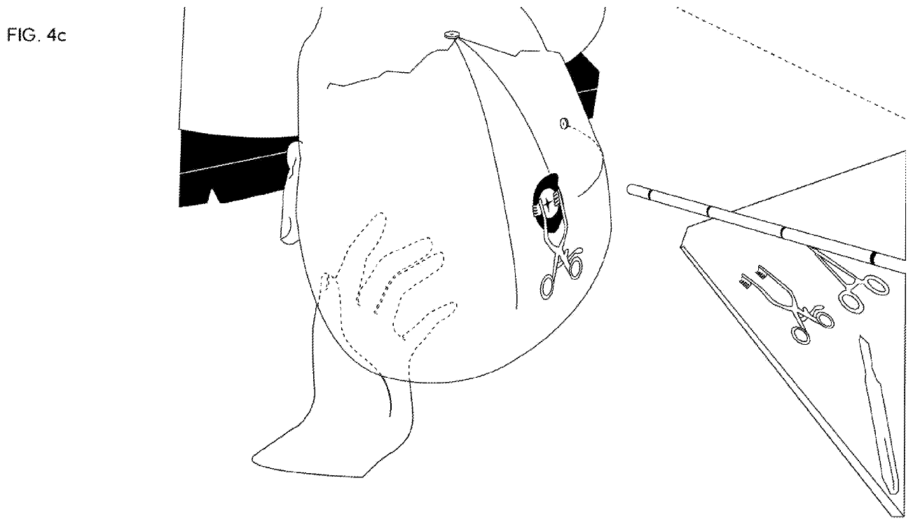

FIGS. 4A, 4B, and 4C are exemplary views showing a preparation for virtual surgery using an MR device according to the present embodiment.

The first and second MR devices 110 and 120 may provide various virtual surgical environments before an actual surgery while the patient's anatomical structure is visualized.

As shown in FIG. 4A, the first MR device 110 may output a menu screen on which a simulation for training is selected in a virtual environment according to an operation or command of an evaluator (an operating surgeon with a high surgical skill) who is equipped with the device.

As shown in FIG. 4B, the first MR device 110 may output a menu screen on which an instrument to be used for training is selected in a virtual environment according to an operation or command of an evaluator (an operating surgeon with a high surgical skill) who is equipped with the device.

As shown in FIG. 4C, the first MR device 110 may perform a virtual surgery corresponding to educational content by using an instrument selected in a virtual environment according to an operation or command of an evaluator (an operating surgeon with a high surgical skill) who is equipped with the device.

The first MR device 110 may repeatedly perform a simulation for educational content according to an operation or command of an evaluator (n operating surgeon with a high surgical skill) who is equipped with the device and thus allow the virtual surgical evaluation apparatus 130 to collect center points (x, y, z), angles, speeds, and the like for each main point, so that an evaluation index table may be generated according to an average.

The virtual surgery evaluation apparatus 130 may obtain evaluator virtual surgery simulation data of an operating surgeon (evaluator) with a high surgical level from the first MR device 110. The virtual surgery evaluation apparatus 130 may extract, from the evaluator virtual surgery simulation data, a preset evaluator-used surgical instrument parameter (e.g., at least one or more of a surgical instrument's entry position (center point), entry angle, entry direction, speed, delay time, and action) for each surgical instrument used by the operating surgeon in an image. The virtual surgery evaluation apparatus 130 may generate an evaluation index table for educational virtual simulation based on a plurality of evaluator-used surgical instrument parameters (e.g., at least one or more of a surgical instrument's entry position (center point), entry angle, entry direction, speed, delay time, and action).

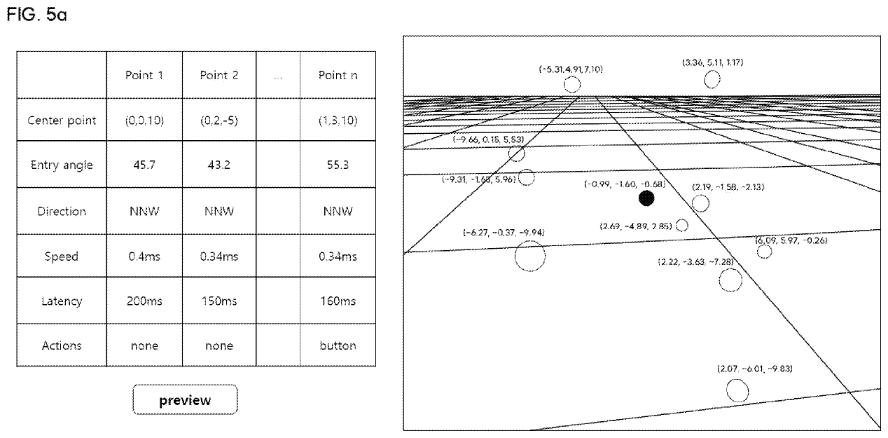

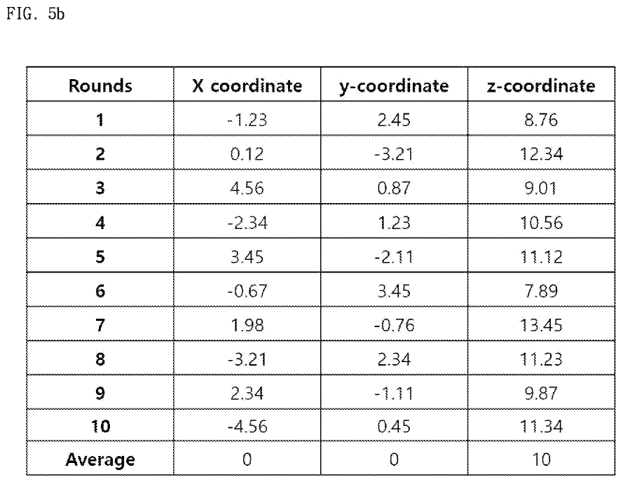

FIGS. 5A and 5B are views showing an evaluation index table according to the present embodiment.

As shown in FIG. 5A, the virtual surgery evaluation apparatus 130 may collect at least one or more of a surgical instrument's entry position (center point), entry angle, entry direction, speed, delay time, and action for each virtual surgical process (first point, second point, and N^{th} point).

The virtual surgery evaluation apparatus 130 may calculate a center point by x, y, and z values of each coordinate of the surgical instrument based on the input evaluator virtual surgery simulation data, and calculate an average center point, an average entry angle/direction, an average speed, and an average delay time by dividing the resulting center point by a total number of coordinates (n times). The virtual surgery evaluation apparatus 130 may automatically set a threshold based on a value farthest from a center value to automatically divide and generate a differential evaluation index table. The virtual surgery evaluation apparatus 130 may generate a center point virtual action of a perfect value based on calculation results, and may automatically generate an evaluation index table with n points based on the center point virtual action of the perfect value. The virtual surgery evaluation apparatus 130 may enable the evaluator to modify and reflect a weight of evaluation items in the automatically generated evaluation index table, and may finally complete the evaluation index table.

As shown in FIG. 5B, the virtual surgery evaluation apparatus 130 may generate reference coordinates for a first point during a surgical process based on the input evaluator virtual surgery simulation data.

The virtual surgery evaluation apparatus 130 may collect x, y, and z coordinates, which are positions for entering a surgical instrument for first to tenth times, with regard to a first point from the evaluator virtual surgery simulation data input during the first to tenth times.

The virtual surgery evaluation apparatus 130 may calculate an average value of the x, y, and z coordinates, which are positions for entering a surgical instrument input during the first to tenth times, to calculate a first point reference table as (0, 0, 10), and calculate a first point maximum distance as “maxProximityDistance=10.”

For example, the virtual surgery evaluation apparatus 130 may generate a first point proximity evaluation code as “proximityPercentage=Mathf.Clamp01 (1f-(distance/maxProximityDistance))×100f’.

For example, when an input coordinate of the subject is (1.9,−1, 9.7) in a first point reference coordinate (0,0,10), the virtual surgery evaluation apparatus 130 may evaluate the proximity as “Proximity Percentage: 80.05006%’.

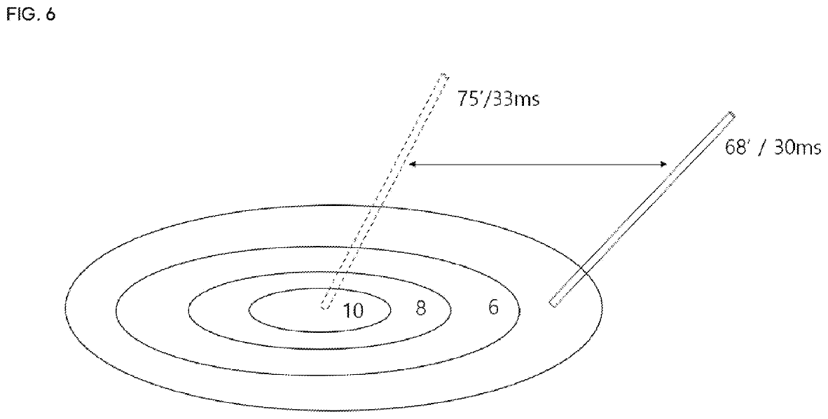

FIG. 6 is an exemplary view of comparing an evaluation index table with a parameter of a surgical instrument used by a subject according to the present embodiment.

As shown in FIG. 6, the virtual surgery evaluation apparatus 130 may divide the subject virtual surgery simulation data according to a surgical process, and then output a result of comparing at least one of a surgical instrument's entry position (center point) 10, entry angle, entry direction, speed (33 ms), delay time, and action corresponding to the evaluation index table with at least one of an entry position (center point) 5, entry angle, entry direction, speed (30 ms), delay time, and action, which are subject-used surgical instrument parameters.

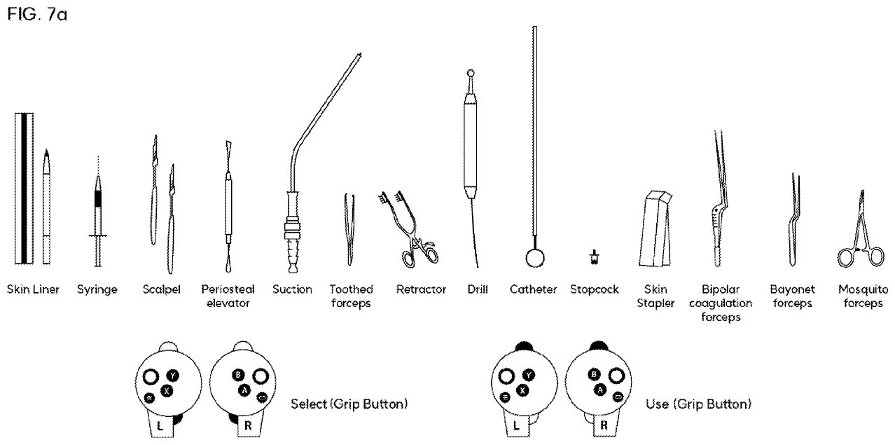

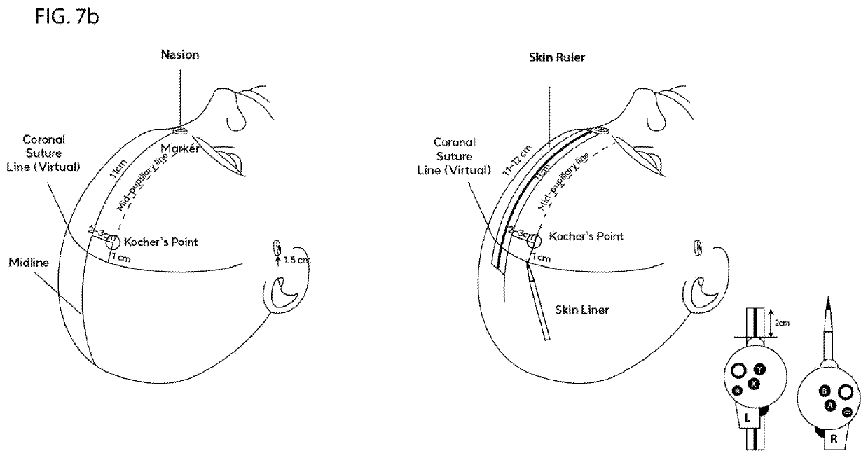

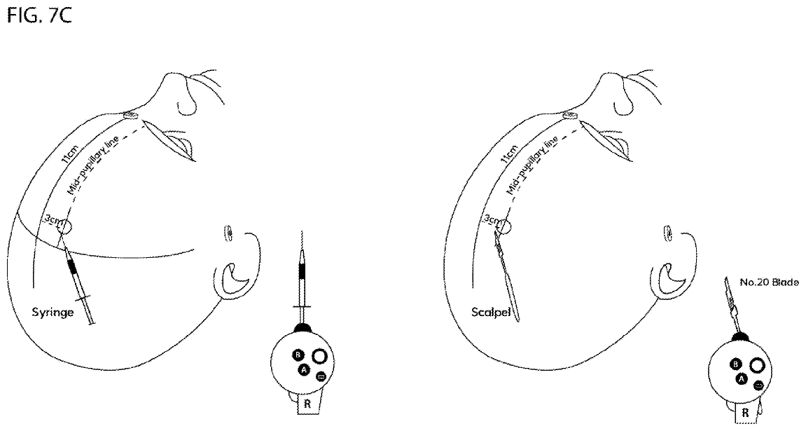

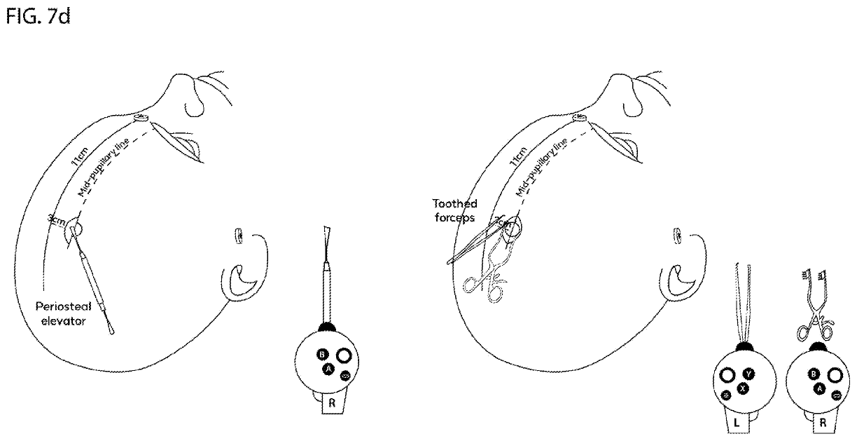

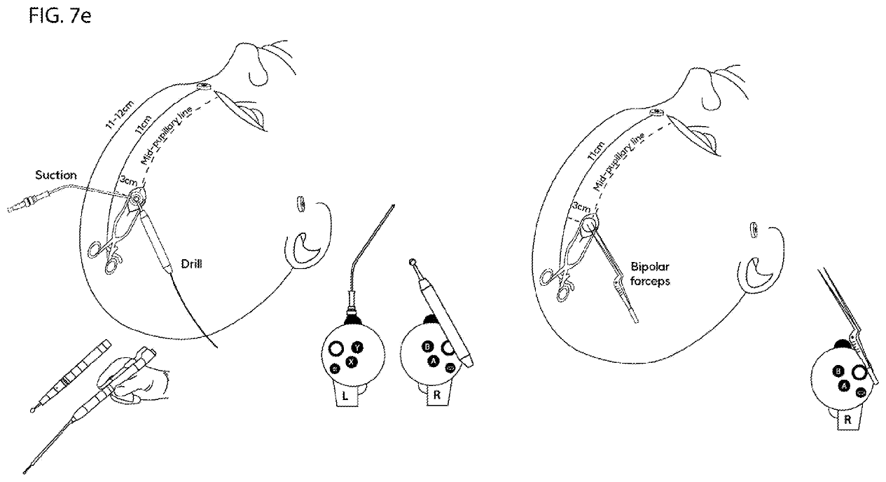

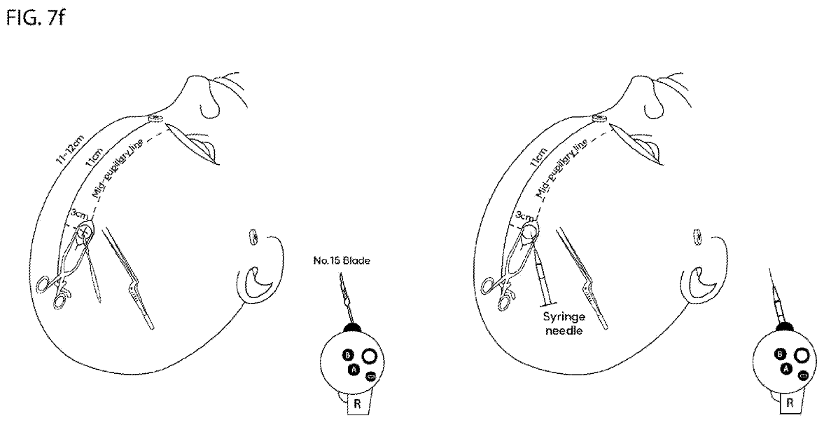

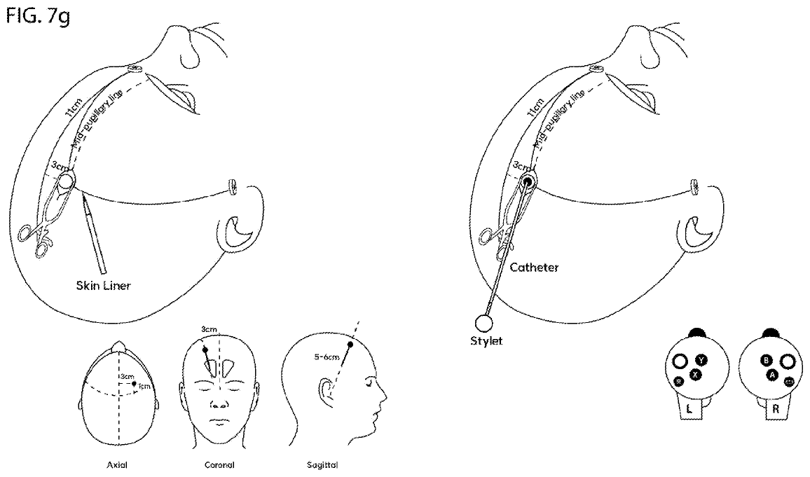

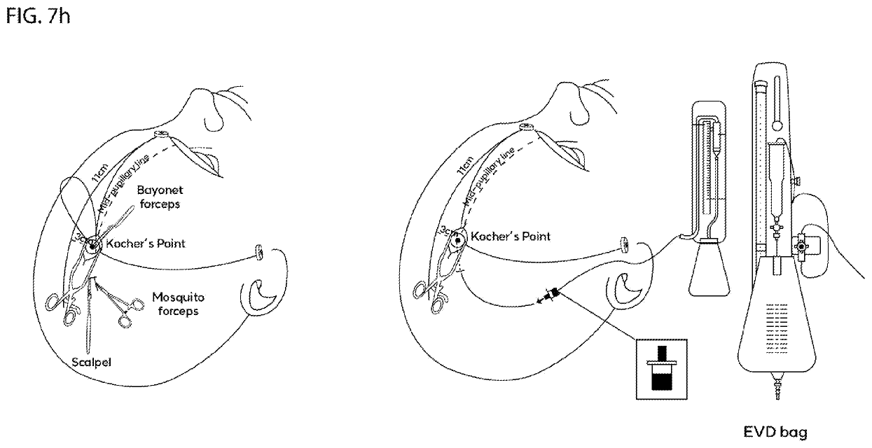

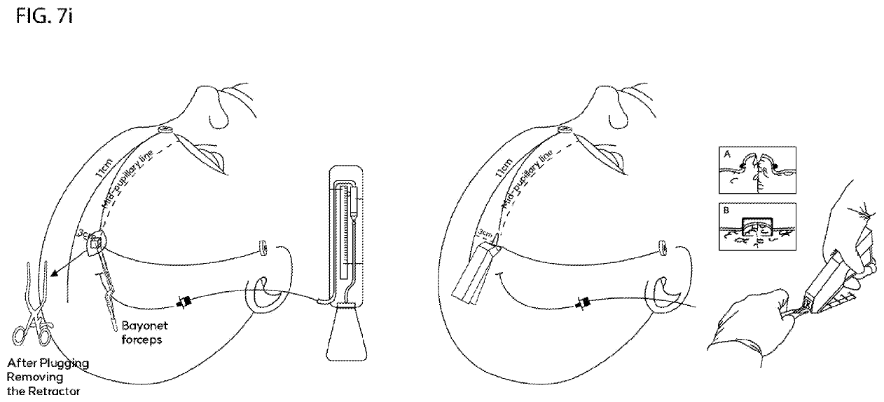

FIGS. 7A, 7B, 7C, 7D, 7E, 7F, 7G, 7H, and 7I are exemplary views showing a virtual surgery using a device according to the present embodiment.

The first and second MR devices 110 and 120 may list a plurality of surgical instruments required for virtual surgery, and select any one of the plurality of surgical instruments according to an operation or command of an operating surgeon (evaluator) or a surgeon-in-training (subject) who performs a virtual surgery.

The first and second MR devices 110 and 120 may generate evaluator virtual surgery simulation data and subject virtual surgery simulation data with respect to the operating surgeon (evaluator) or the surgeon-in-training (subject) and transmit the same to the virtual surgery evaluation apparatus 130.

The virtual surgery evaluation apparatus may be 130 preferably applied to a surgery called an external ventricular drain (EVD), but is not limited thereto, and may also be applied to other surgeries other than the EVD.
1. The virtual surgery evaluation apparatus 130 may classify a process of attaching a marker to a Nasion and EAM anterior 1 cm point to display a position guide in which a catheter is to be inserted while a patient's anatomical structure is visualized for an EVD surgery in the surgeon-in training (subject) virtual surgery image received from the first and second MR devices 110 and 120 as a Kocher's point determination process.
2. The virtual surgery evaluation apparatus 130 may classify a process of displaying a vertical incision line and then performing a dressing process in the surgeon-in-training (subject) virtual surgery images received from the first and second MR devices 110 and 120 as a process of representing a skin liner.
3. The virtual surgery evaluation apparatus 130 may classify a process of administering an anesthetic injection to a lidocaine incision position and a subcutaneous tissue in the surgeon-in-training (subject) virtual surgery image received from the first and second MR devices 110 and 120 as a process of representing a 2% lidocaine injection.
4. The virtual surgery evaluation apparatus 130 may classify a process of performing a vertical incision with a No.20 Blade in the surgeon-in-training (subject) virtual surgery image received from the first and second MR devices 110 and 120 as a process of representing a vertical incision.
5. The virtual surgery evaluation apparatus 130 may classify a process of peeling off periosteal with a periosteal elevator in the surgeon-in-training (subject) virtual surgery image received from the first and second MR devices 110 and 120 as processes of representing a periosteum separation.
6. The virtual surgery evaluation apparatus 130 may classify a process of fixing an incision site using toothed forceps and retractor in the surgeon-in-training (subject) virtual surgery image received from the first and second MR devices 110 and 120 as a process of representing a retractor fixation.
7. The virtual surgery evaluation apparatus 130 may classify a drilling process in the surgeon-in-training (subject) virtual surgery image received from the first and second MR devices 110 and 120 as a process of representing drilling.
8. The virtual surgery evaluation apparatus 130 may classify a process of coagulating the dura with bipolar forceps in the surgeon-in-training (subject) virtual surgery image received from the first and second MR devices 110 and 120 as a process of representing coagulating dura.
9. The virtual surgery evaluation apparatus 130 may classify a process of incising the dura with a No. 15 Blade in the form of cross (+) and coagulating the dura which is incised by bipolar forceps in the surgeon-in-training (subject) virtual surgical image received from the first and second MR devices 110 and 120 as a process of representing a cross-formed incision of the dura.
10. The virtual surgery evaluation apparatus 130 may classify a process of incising a brain cortex into a small size of a cross (+) with a syringe needle to make an opening through which a catheter may enter and coagulating a cortex opening site with a bipolar in the surgeon-in-training (subject) virtual surgery image received from the first and second MR devices 110 and 120 as a process of representing a brain cortex.
11. The virtual surgery evaluation apparatus 130 may classify a process of displaying a trajectory in the surgeon-in-training (subject) virtual surgery image received from the first and second MR devices 110 and 120 as a process of representing a trajectory.
12. The virtual surgery evaluation apparatus 130 may classify a process of inserting a catheter into a 5 cm depth, removing a stylet, and checking whether the cerebrospibal fluid (CSF) is drained in the surgeon-in-training (subject) virtual surgery image received from the first and second MR devices 110 and 120 as a process of inserting a catheter.
13. The virtual surgery evaluation apparatus 130 may classify a process of incising an exit to be tunneled about 4-5 cm on a subcutaneous layer with No. 15 Blade, holding a catheter with bayonet forceps, and removing a catheter distal part using mosquito forceps to take the same out of the skin as a process of representing tunneling.
14. The virtual surgery evaluation apparatus 130 may classify a process of connecting a drain cock and an EVD bag to a catheter distal end in the surgeon-in-training (subject) virtual surgery image received from the first and second MR devices 110 and 120 as a process of representing a connection of a drain cock and an EVD bag.
15. The virtual surgery evaluation apparatus 130 may classify a process of plugging a burr hole with a gelform sized 1.5 cm×1.5 cm in the surgeon-in-training (subject) virtual surgery image received from the first and second MR devices 110 and 120 as a process of representing a plugging burr hole.
16. The virtual surgery evaluation apparatus 130 may classify a process of plugging a burr hole with a gelform sized 1.5 cm×1.5 cm in the surgeon-in-training (subject) virtual surgery image received from the first and second MR devices 110 and 120 as a process of representing a skin stapler.

The above description is merely illustrative of the technical idea of the present embodiment, and those skilled in the art to which the present embodiment pertains may make various changes and modifications without departing from the essential characteristics of the present embodiment. Thus, the present embodiments are not intended to limit, but intended to explain the technical idea of the embodiments of the present invention, and the scope of the technical idea of the present embodiment is not limited thereto. The scope of protection of the present embodiment should be interpreted according to the following claims, and all technical ideas within the scope equivalent thereto should be interpreted as being included in the scope of rights of the present embodiment.

What is claimed is:

1. An apparatus for evaluating a virtual surgery, the apparatus comprising:
an evaluator simulation data acquisition unit for obtaining an evaluator's evaluator virtual surgery simulation data from a first MR device;
an evaluation index table generation unit for extracting a preset evaluator-used surgical instrument parameter for each surgical instrument used by the evaluator based on the evaluator virtual surgery simulation data, and generating an evaluation index table based on the used surgical instrument parameter;
a subject simulation data acquisition unit for obtaining a subject's subject virtual surgery simulation data from a second MR device; and
a virtual surgery evaluation unit for extracting a preset subject-used surgical instrument parameter for each surgical instrument used by the subject based on the subject virtual surgery simulation data, and comparing the evaluation index table with the subject-used surgical instrument parameter to evaluate a subject's skill level.

2. The apparatus of claim 1, wherein the first MR device
enters an evaluation index table generation mode in a virtual environment by the evaluator's operation or command, outputs a target surgery name for generating the evaluation index table and a virtual object corresponding to a surgical instrument, allows the surgical instrument to be gripped using a virtual hand gesture, and
generates the evaluator virtual surgery simulation data when a corresponding surgery is completed after allowing each surgical procedure to be performed by hand gesture according to a simulation action of the surgical instrument while an anatomical structure of a patient corresponding to the surgery name is visualized.

3. The apparatus of claim 2, wherein the evaluation index table generation unit
generates the evaluation index table based on the used surgical instrument parameter by recognizing that for generating an evaluation table for a virtual surgery is accumulated when a corresponding surgery is repeated by a preset number of times (n times) after allowing each surgical procedure to be performed on a simulation action for the target surgery name which is identical to the operation or command of the evaluator from the first MR device.

4. The apparatus of claim 1, wherein the evaluation index table generation unit
divides the evaluator virtual surgery simulation data into a plurality of steps, and extracts the evaluator-used surgical instrument parameter comprising at least one or more of a surgical instrument's entry position (center point), entry angle, entry direction, speed, delay time, and action for each surgical instrument used by the evaluator in each step.

5. The apparatus of claim 4, wherein the evaluation index table generation unit
generates the evaluation index table based on an average value for at least one or more of a surgical instrument's entry position (center point), entry angle, entry direction, speed, delay time, and action for each surgical instrument used by the evaluator in each step.

6. The apparatus of claim 1, wherein the second MR device
enters a skill level evaluation mode in a virtual environment by the subject's operation or command, outputs a target surgery name for evaluating a skill level and a virtual object corresponding to the surgical 1 instrument, allows the surgical instrument to be gripped using a virtual hand gesture, and
generates the subject virtual surgery simulation data when a corresponding surgery is completed after allowing each surgical procedure to be performed by hand gesture according to a simulation action of the surgical instrument while an anatomical structure of a patient corresponding to the surgery name is visualized.
